(19) United States
(12) Patent Application Publication
(10) Pub. No.: US 2024/0318254 A1
(43) Pub. Date: Sep. 26, 2024

(54) CONSTRUCTION METHOD OF RISK PREDICTION MODEL FOR PROGNOSIS OF GASTRIC CANCER

(71) Applicant: Tangshan People's Hospital, Tangshan City (CN)

(72) Inventors: Xuan ZHENG, Tangshan City (CN); Jingwu LI, Tangshan City (CN); Jiangong WANG, Tangshan City (CN); Yufeng LI, Tangshan City (CN); Zhiwu WANG, Tangshan City (CN); Qingke LI, Tangshan City (CN); Lei WANG, Tangshan City (CN); Dan LI, Tangshan City (CN); Zhuo WANG, Tangshan City (CN); Jun ZHANG, Tangshan City (CN)

(21) Appl. No.: 18/217,921

(22) Filed: Jul. 3, 2023

(30) Foreign Application Priority Data
Mar. 22, 2023 (CN) 2023102806825

(51) Int. Cl. C12Q 1/6886

(52) U.S. Cl. CPC C12Q 1/6886; G01N 2800/50; G01N 2800/7028

(57) ABSTRACT

The present disclosure provides a construction method of a risk prediction model for a prognosis of gastric cancer, and belongs to the technical field of biomedicine. In the present disclosure, from the perspective of bioinformatics analysis, it is predicted that gastric cancer exosomes carry circRNAs. Markers regulating the occurrence and development of gastric cancer are selected by combining the circRNAs with an RNA binding protein (RBP). A predictive marker for the prognosis of gastric cancer is an RPB gene, including one or more of AUH, HNRNPC, HNRNPD, U2AF2, and FXR1. The risk prediction model constructed based on the predictive marker can quickly and accurately predict the prognosis of patients with gastric cancer.

### CROSS REFERENCE TO RELATED APPLICATION

This patent application claims the benefit and priority of Chinese Patent Application No. 2023102806825, filed with the China National Intellectual Property Administration on Mar. 22, 2023, the disclosure of which is incorporated by reference herein in its entirety as part of the present application.

### TECHNICAL FIELD

The present disclosure belongs to the technical field of biomedicine, and in particular relates to a construction method of a risk prediction model for a prognosis of gastric cancer.

### BACKGROUND

Gastric cancer is one of the major problems affecting human health worldwide, especially in East Asian countries such as Korea, Japan, and China. Despite advances in endoscopic and surgical resection equipment and techniques, gastric cancer is characterized by rapid progression to advanced stage and high metastasis, and patients with metastatic gastric cancer show an extremely poor prognosis. Currently, gastric cancer remains the third most common cause of cancer-related death.

In recent years, with the advancement and application of research technologies, especially high-throughput sequencing, researchers have identified a large number of circular RNAs (circRNAs) in eukaryotic transcriptomes. Some of these circRNAs are 10-fold more abundant than their linear cognates. The role of the circRNA in tumorigenesis and the potential of the circRNA as a novel clinical diagnostic marker have gradually been recognized. Evidence has shown that circRNAs play a role in the development of gastric cancer. However, the mechanism of circRNAs in the development of gastric cancer is not fully understood. At present, the research on functions of the circRNA is mainly divided into the following types. The circRNAs can act as miRNA sponges and regulate miRNA-mediated gene silencing. Some circRNAs containing internal ribosome entry sites can encode novel proteins or peptides. In addition, some circRNAs can interact with RNA binding proteins (RBPs) to regulate gene expression. Since there are relatively few known RBPs that can bind to the circRNA, while a specific mechanism has not been fully elucidated. Therefore, it is difficult to screen RBPs that are closely related to the occurrence and development of gastric cancer. So far, there has been no relevant report on exploring the occurrence and development of gastric cancer from the perspective of gastric cancer exosomes carrying circRNAs and combining with RBPs.

### SUMMARY

The present disclosure provides a construction method of a risk prediction model for a prognosis of gastric cancer. This method can quickly and accurately evaluate the prognosis of patients with gastric cancer.

To solve the above technical problems, the present disclosure provides the following technical solutions:

The present disclosure provides a construction method of a risk prediction model for a prognosis of gastric cancer, including the following steps: (1) collecting differentially-expressed circRNAs from gastric cancer cell-derived exosomes and gastric cancer tissues; (2) screening an RBP gene that is differentially expressed in the gastric cancer tissues and targetedly binded with the differentially-expressed circRNAs; (3) obtaining a prognostic RBP gene with a screening criterion of p value<0.05 for the differentially-expressed RBP gene and according to the differentially-expressed RBP gene and a proportional hazards (Cox) regression model; (4) calculating a Risk score of each sample of the gastric cancer tissues according to an expression level of the prognostic RBP gene and a regression coefficient corresponding to the prognostic RBP gene; and (5) based on the Risk score of each sample of the gastric cancer tissues, calculating a median value for each sample of the gastric cancer tissues, and classifying each sample of the gastric cancer tissues into a high-risk group and a low-risk group according to the median value.

Preferably, the prognostic RBP gene includes one or more ofAUH, HNRNPC, HNRNPD, U2AF2, and FXRL.

Preferably, a process of collecting the differentially-expressed circRNAs includes: screening the differentially-expressed circRNAs in the gastric cancer cell-derived exosomes from a data set GSE202538 and the gastric cancer tissues from a data set GSE83521 with an R language package and screening criteria of |log FC|>1 and p value<0.05 for a differential gene.

Preferably, a process of screening the differentially-expressed RBP gene that is targetedly binded with the differentially-expressed circRNAs includes: (1) predicting RBPs that are targetedly binded with the circRNAs through three databases of Starbase, CSCD, and Circinteractome, and taking a union of the RBPs in the three databases to obtain an RBP that is targetedly binded with the circRNAs; and (2) screening the RBP that is targetedly binded with the circRNAs obtained in step (1) in a data set of a gastric cancer transcriptome of a database TCGA with an R language package, and screening the differentially-expressed RBP gene with screening criteria of |log FC|>0.8 and p value<0.05 for same differential genes.

Preferably, a calculation method of the Risk score is shown in formula (1) as follows:

$$Risk score = {\sum }_{i = 1}^{n} {coef}_{i} \times {exp}_{i} ,$$

n represents a number of the prognostic RBP gene; coefᵢ represents a regression coefficient of a prognostic RBP gene i; and expᵢ represents an expression level of the prognostic RBP gene i.

Preferably, if the Risk score is less than the median value, a test sample of the gastric cancer tissues is of low risk, indicating that a gastric cancer patient has a desirable prognosis; and if the Risk score is greater than or equal to the median value, the test sample of the gastric cancer tissues is of high risk, indicating that the gastric cancer patient has a poor prognosis.

Preferably, based on the Risk score obtained in the risk prediction model and clinical data of a patient, a receiver operator characteristic (ROC) curve is plotted for the risk prediction model and clinical characteristics including age, gender, T staging, and N staging; and an accuracy of the risk prediction model for a prognosis in predicting the prognosis of a gastric cancer patient is evaluated according to an area under the curve (AUC).

The present disclosure further provides use of a risk prediction model obtained by the construction method in risk evaluation for a prognosis of gastric cancer.

Compared with the prior art, the present disclosure has the following beneficial effects:

In the present disclosure, for the first time, a risk prediction model for a prognosis of gastric cancer is constructed using AUH, HNRNPC, HNRNPD, U2AF2, and FXR1 as prognostic markers. The risk prediction model obtained by the construction method can quickly and accurately evaluate the prognosis of gastric cancer patients, to determine the prognostic risk of gastric cancer patients. This is conducive to the allocation of medical resources, the formulation of appropriate treatment plans, and the guidance of individualized treatment, and has a desirable clinical application prospect.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-E show GEO database analysises of differential expression of the circRNAs in gastric cancer cell-derived exosomes and gastric cancer tissues (where FIG. 1A is a volcano plot of differential expression of the circRNAs in the gastric cancer cell-derived exosomes in a data set GSE202538, Normal-exo: n=1, GC-exo: n=4, red dots represent highly-expressed genes, green dots represent low-expressed genes, a vertical axis represents a 2-based logarithm value of the fold change (FC) between different groups, that is, log 2 (Fold Change), and a horizontal axis represents a −10-based logarithm value of the P value of a significant difference test, namely −log 10(p-value); FIG. 1B is a volcano plot of differential expression of the circRNAs in the gastric cancer tissues of a data set GSE83521, Normal: n=6, GC: n=6; FIG. 1C is an intersection Venn diagram of differentially-expressed circRNAs in the gastric cancer cell-derived exosomes and the gastric cancer tissues; FIG. 1D is a differential expression heat map of the circRNAs in the data set GSE202538, Normal-exo: n=1; GC-exo: n=4; and E is a differential expression heat map of the circRNAs in the data set GSE83521, Normal: n=6; GC: n=6);

FIGS. 2A-D show the prediction of circRNA-RBP binding and the analysis of differential expression of the RBP in gastric cancer (where FIG. 2A is an interaction network between the circRNAs and RBP plotted by Cytoscape software, red triangles represent the circRNAs, yellow circles represent the RBPs, and gray lines represent the interaction; FIG. 2B is a differential expression volcano plot of the RBP gene in a gastric cancer data set of the database TCGA, Normal: n=32, GC: n=375; FIG. 2C is the enrichment of differential RBP genes in a biological process obtained by GO functional analysis; and FIG. 2D is a KEGG pathway enrichment analysis of the differential RBP genes);

FIGS. 3A-I show risk models of the association between the RBP and the prognosis of patients with gastric cancer (where FIG. 3A is a forest plot of a univariate COX regression analysis, the abscissa indicates Hazard Ratio, the ordinate indicates the gene name, risk value, and p value, green represents low risk factors, and red represents high risk factors; FIG. 3B is a forest plot of a multivariate COX regression analysis; FIG. 3C is the grouping of sample risk values in the risk model, the abscissa indicates the order of samples, the ordinate indicates the risk value, and the median value is a dividing line to sort the samples according to the risk value from high to low; FIG. 3D is the survival distribution of patients in the high-risk group and low-risk group, the abscissa indicates the order of the samples, the ordinate indicates the survival time, the green dots indicate surviving samples, and the red dots indicate dead samples; FIG. 3E is an expression heat map of key RBP genes in the risk model; FIG. 3F is a Kaplan-Meier survival curve analysis of high-risk and low-risk patients; FIG. 3G is a forest plot of a univariate independent prognostic analysis; FIG. 3H is a forest plot of a multivariate independent prognostic analysis; and FIG. 3I is a ROC analysis of the risk model and related clinical characteristics, and the lower right corner is an AUC; the number of samples: n=433); and

FIGS. 4A-L show correlation analysises between clinicopathological features and the prognosis of patients with gastric cancer (where FIGS. 4A-D are Kaplan-Meier survival curve analysises of the relationship between the age, gender, T staging, and N staging and the survival of gastric cancer patients; FIGS. 4E-L are predictive abilities of the risk model constructed based on the Kaplan-Meier survival curve analysis and verification for patients with different ages, genders, T staging, and N staging).

### DETAILED DESCRIPTION OF THE EMBODIMENTS

The present disclosure provides a construction method of a risk prediction model for a prognosis of gastric cancer, including the following steps: (1) collecting differentially-expressed circRNAs from gastric cancer cell-derived exosomes and gastric cancer tissues; (2) screening an RBP gene that is differentially expressed in the gastric cancer tissues and targetedly binded with the differentially-expressed circRNAs; (3) obtaining a prognostic RBP gene with a screening criterion of p value<0.05 for the differentially-expressed RBP gene and according to the differentially-expressed RBP gene and a proportional hazards (Cox) regression model; (4) calculating a Risk score of each sample of the gastric cancer tissues according to an expression level of the prognostic RBP gene and a regression coefficient corresponding to the prognostic RBP gene; and (5) based on the Risk score of each sample of the gastric cancer tissues, calculating a median value for each sample of the gastric cancer tissues, and classifying each sample of the gastric cancer tissues into a high-risk group and a low-risk group according to the median value. In the present disclosure, the prognostic RBP gene includes one or more of AUH, HNRNPC, HNRNPD, U2AF2, and FXR1. The AUH, HNRNPC, HNRNPD, and U2AF2 are low risk factors, and the FXR1 is a high risk factor. The prognostic genes mainly play a role in the regulation of mRNA metabolic processes, mRNA processing, and RNA splicing.

In the present disclosure, a process of collecting the differentially-expressed circRNAs includes: screening the differentially-expressed circRNAs in the gastric cancer cell-derived exosomes from a data set GSE202538 and the gastric cancer tissues from a data set GSE83521 with an R language package and screening criteria of |log FC|>1 and p value<0.05 for a differential gene. The R language package includes a “limma” package, a “ggplot2” package, and a “heatmap” package. The data set GSE202538 includes 1 human normal gastric epithelial exosome sample and 4 different gastric cancer cell exosome samples. The data set GSE83521 includes 6 gastric cancer tissue samples and 6 adjacent normal mucosal tissue samples. Both the data sets GSE202538 and GSE83521 are downloaded from a database Gene Expression Omnibus (GEO) (https://www.ncbi.nlm.nih.gov/gds).

In the present disclosure, a process of screening the differentially-expressed RBP gene that is targetedly binded with the differentially-expressed circRNAs includes: (1) predicting RBPs that are targetedly binded with the circRNAs through three databases of Starbase (https://starbase.sysu.edu.cn/), Cancer Specific circRNA Database (CSCD) (gb.whu.edu.cn/CSCD/), and Circinteractome (https://circinteractome.nia.nih.gov/index.html), and taking a union of the RBPs in the three databases to obtain an RBP that is targetedly binded with the circRNAs; and (2) screening the RBP that is targetedly binded with the circRNAs obtained in step (1) in a data set of a gastric cancer transcriptome of a database TCGA with an R language package, and screening the differentially-expressed RBP gene with screening criteria of |log FC|>0.8 and p value<0.05 for a differential gene. A regulatory network of the circRNA and RBP is plotted by Cytoscape (v3.6.0) software for the combined RBPs in the three databases. The RNA-seq data (HTSeq-FPKM) of 375 gastric cancer tissue samples and 32 adjacent normal tissue samples are downloaded from a database The Cancer Genome Atlas (TCGA) (https://portal.gdc.cancer.gov/). In step (2), the R language package includes a “limma” package, a “impute” package, and a “ggplot2” package. GO function analysis and KEGG pathway enrichment analysis are further conducted on the selected differentially-expressed RBP gene to determine that the differentially-expressed RBP gene can mainly play a role in the regulation of mRNA metabolic processes, mRNA processing, and RNA splicing.

In the present disclosure, based on the gastric cancer-related data set GSE84437, a “survival” package of the R language is used to conduct univariate COX analysis on the differentially-expressed RBP gene. With P<0.05 as a screening criterion of the differentially-expressed RBP gene, the RBP genes related to prognosis are identified. Multivariate COX analysis is conducted, and RBP genes with P<0.05 are selected as markers for the prognostic risk model. Meanwhile, a regression coefficient (coef) of the RBP gene corresponding to P<0.05 is obtained from the multivariate COX analysis, and a prognostic risk model is constructed. Based on the constructed model, a Risk score of each patient is calculated to obtain a median value. The data set GSE84437 includes 433 gastric cancer tissue samples, and is downloaded through the database Gene Expression Omnibus (GEO) (https://www.ncbi.nlm.nih.gov/gds). The median value is calculated based on the Risk score of each of the 433 gastric cancer tissue samples.

In the present disclosure, a calculation method of the Risk score in the risk model is shown in formula (1) as follows:

$$Risk score = {\sum }_{i = 1}^{n} {coef}_{i} \times {exp}_{i} ,$$

where
n represents a number of the prognostic RBP gene; coefᵢ represents a regression coefficient of a prognostic RBP gene i; and expᵢ represents an expression level of the prognostic RBP gene i. Based on the selected prognostic RBP genes, a calculation method of the Risk score is shown in formula (2) as follows: Risk score=(−0.25747×expression level ofAUH)+(−0.53445×expression level of HNRNPC)+(−0.44937×expression level of HNRNPD)+(−0.3861×expression level of U2AF2)+(0.554848×expression level of FXR1).

In the present disclosure, if the Risk score is less than the median value, a test sample of the gastric cancer tissues is of low risk, indicating that a gastric cancer patient has a desirable prognosis; and if the Risk score is greater than or equal to the median value, the test sample of the gastric cancer tissues is of high risk, indicating that the gastric cancer patient has a poor prognosis. The median value is 0.98541, calculated based on the 433 gastric cancer tissue samples and the risk model.

In the present disclosure, an independent prognostic analysis is conducted on the constructed risk model: based on the Risk score obtained in the risk model and the clinical data of the patient, the univariate and multivariate independent prognostic analysis are conducted using the R language “survival” package, and the ROC analysis is conducted using the R language “survivalROC” package; a ROC curve is plotted for the risk prediction model and clinical characteristics including age, gender, T staging, and N staging; and an accuracy of the risk prediction model for a prognosis in predicting the prognosis of a gastric cancer patient is evaluated according to an AUC. Further, the “survminer” and “survival” packages of the R language are used to analyze the relationship between the age, gender, T staging, and N staging and the survival of patients with gastric cancer, and to verify the predictive ability of the risk model constructed in predicting the survival of patients with different ages, genders, T staging, and N staging.

The present disclosure further provides use of a risk prediction model obtained by the construction method in risk evaluation for a prognosis of gastric cancer. In the present disclosure, the risk prediction model can quickly and accurately evaluate the prognosis of gastric cancer patients.

The technical solutions of the present disclosure will be clearly and completely described below with reference to the examples of the present disclosure.

### Example 1

### 1. Acquisition of Samples

Through a database Gene Expression Omnibus (GEO) (https://www.ncbi.nlm.nih.gov/gds), a data set GSE202538 of gastric cancer cell-derived exosome-related circRNAs expression was downloaded, including 1 human normal gastric epithelial cell exosome sample and 4 different gastric cancer cell exosome samples. A data set SE83521 of gastric cancer tissue-related circRNAs expression was downloaded, including 6 gastric cancer tissue samples and 6 adjacent normal mucosa tissue samples. A gastric cancer-related data set GSE84437 was downloaded, including 433 gastric cancer tissue samples.

Through a database The Cancer Genome Atlas (TCGA) (https://portal.gdc.cancer.gov/), RNA-seq data (HTSeq-FPKM) of 375 gastric cancer tissue samples and 32 adjacent normal tissue samples were downloaded.

2. Analysis of Differential Expression of circRNAs and Acquisition of Differentially-Expressed circRNAs

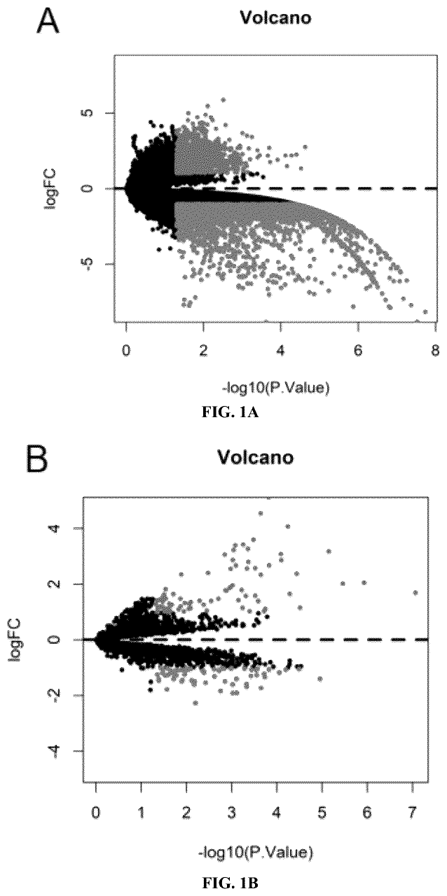

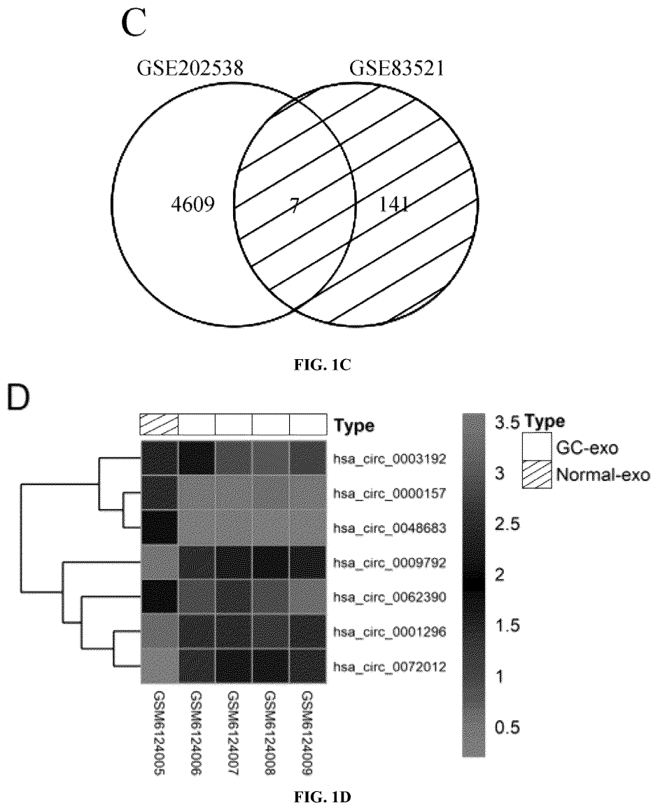

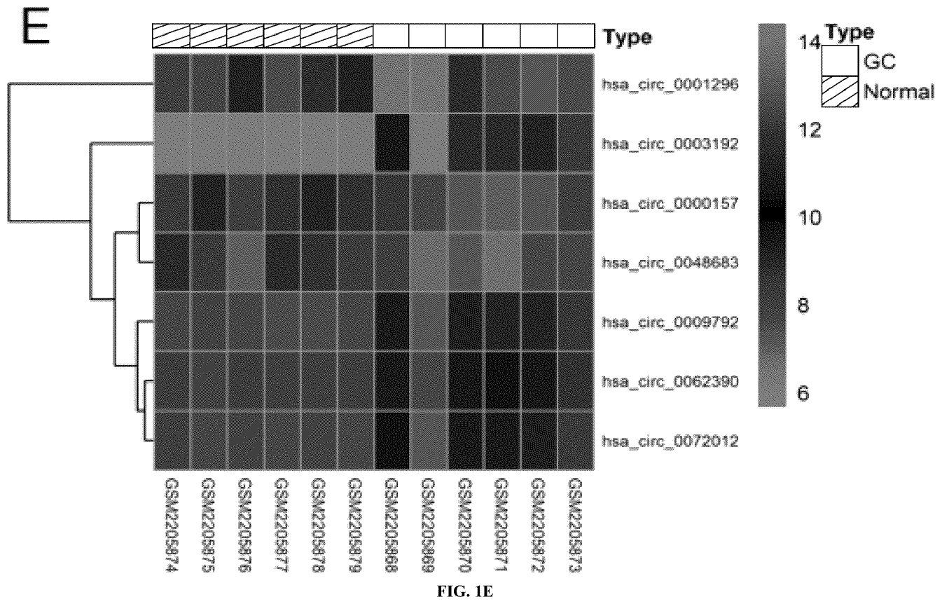

The “limma” package of R language was used to screen the differentially-expressed circRNAs in the exosomes of the dataset GSE202538, and the differentially-expressed circRNAs in the tissues of the dataset GSE83521, with |log FC|>1 and p value<0.05 as the screening criteria of differential genes. The volcano plots FIG. 1A and FIG. 1B of the differentially-expressed circRNAs in gastric cancer cell-derived exosomes and gastric cancer tissues, respectively, and an intersection Venn diagram FIG. 1C of differentially-expressed circRNAs in the gastric cancer cell-derived exosomes and the gastric cancer tissues were plotted with the “ggplot2” software package of R language. The heatmaps FIG. 1D to FIG. 1E of differential expression of the circRNAs were plotted with the “heatmap” package.

As shown in FIGS. 1A-E, in this analysis: a total of 4,616 differentially-expressed circRNAs in gastric cancer cell-derived exosomes were obtained, including 1,112 up-regulated circRNAs and 3,504 down-regulated circRNAs (FIG. 1A). A total of 148 differentially-expressed circRNAs in gastric cancer tissues were obtained, including 82 up-regulated circRNAs and 66 down-regulated circRNAs (FIG. 1B). Taking an intersection of the two, a total of 7 intersection circRNAs (namely same circRNAs) were obtained, namely hsa_circ_0072012, hsa_circ_0001296, circ_0062390, hsa_circ_0003192, hsa_circ 0000157, hsa_circ_0048683, and hsa_circ_0009792 (FIG. 1C). The differential expression heat maps of the 7 intersection circRNAs in the two data sets were shown in FIG. 1D to FIG. 1E.

3. Construction of circRNA-RBP (RNA Binding Protein) Network

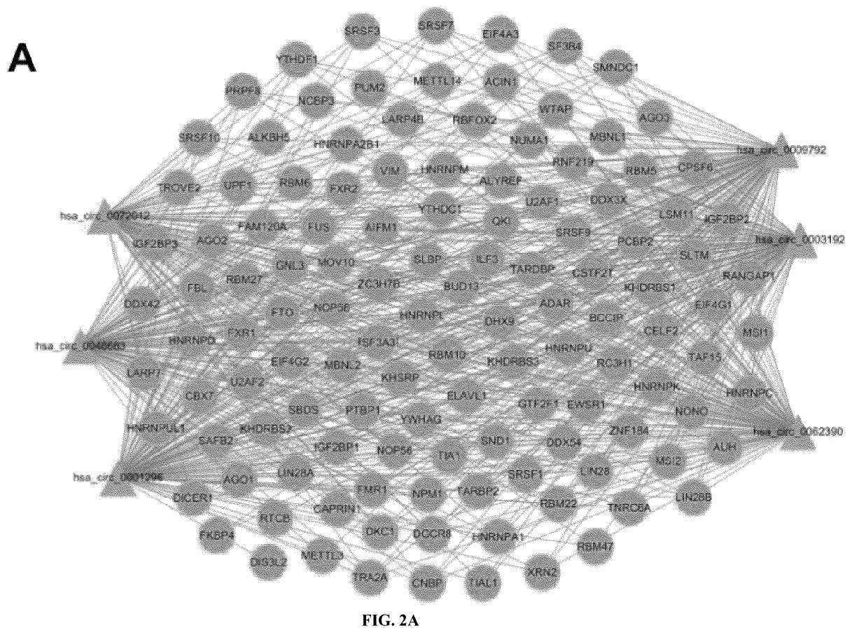

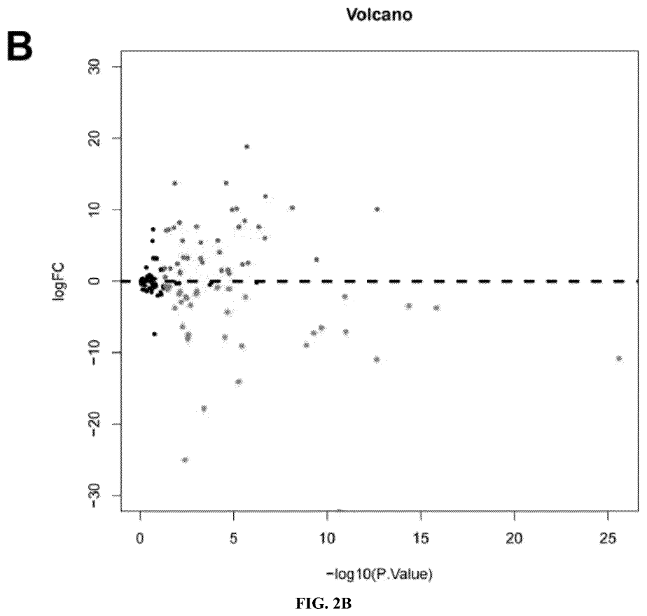

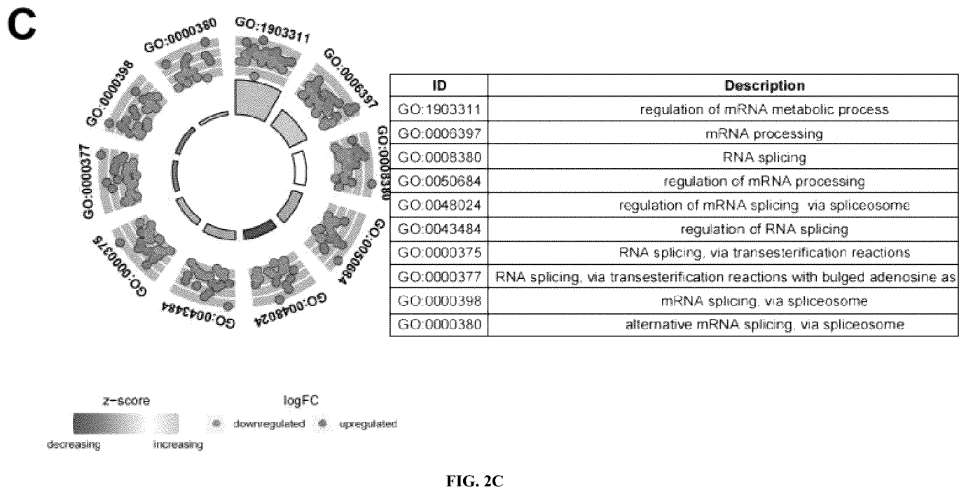

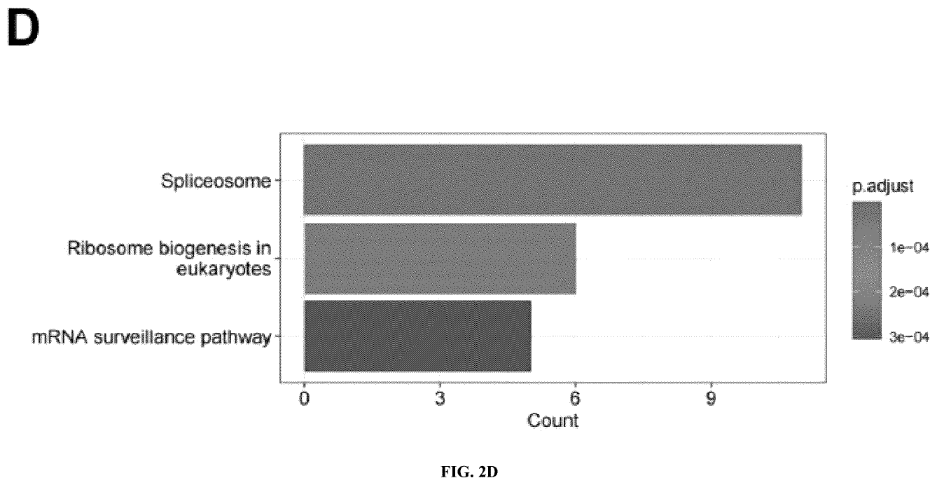

Through three databases StarBase (https://starbase.sysu.edu.cn/), Cancer Specific circRNA Database (CSCD) (gb.whu.edu.cn/CSCD/), and Circinteractome (https://circinteractome.nia.nih.gov/index.html), the 7 RBPs obtained in step 2 that targetedly binded to circRNA were predicted. The union of RBPs predicted by the three databases was taken, and then a regulatory network of circRNA and RBP was plotted by Cytoscape (v3.6.0) software (FIG. 2A).

As shown in FIG. 2A, the combination of 7 circRNAs and RBPs was predicted by Starbase, CSCD, and Circinteractome databases, and a total of 125 RBPs that could be combined with 6 differential circRNAs were obtained (the binding relationship between hsa_circ_0000157 and RBP was not predicted in the three databases), and the interaction network of circRNA-RBP was constructed.

4. Differential Expression Analysis of RBP Genes Binded with Differential circRNAs

The expression data of 125 RBP genes obtained in step 3 of the RNA-seq data (HTSeq-FPKM) of 375 gastric cancer tissue samples and 32 adjacent normal tissue samples downloaded from the gastric cancer data set of the TCGA database were extracted. With |log FC|>0.8 and p value<0.05 as screening criteria of the differential gene, the differentially-expressed RBPs in the gastric cancer transcriptome data set in the TCGA database were selected using the “limma” and “impute” packages of the R language. The expression points of the differential gene were plotted by the “ggplot2” package of R language (FIG. 2B), and 71 differentially-expressed RBP genes were obtained, including 37 significantly up-regulated genes and 34 significantly down-regulated genes.

### 5. GO Function Analysis and KEGG Pathway Enrichment Analysis of Differentially-Expressed RBP Gene

GO (Gene Onotology) analysis and KEGG (Kyoto Encyclopedia of Genes and Genomes) enrichment of differentially-expressed RBP genes were conducted with “clusterProfiler”, “org.Hs.eg.db”, “enrichplot” and “GOplot” packages in the R language, and a circle diagram of the biological process in GO (FIG. 2C) and the histogram of the KEGG enrichment analysis results (FIG. 2D) were plotted, respectively.

As shown in FIG. 2C to FIG. 2D, the results of GO functional enrichment analysis showed that differential genes were mainly enriched in biological processes such as the regulation of mRNA metabolic processes, mRNA processing, and RNA splicing (FIG. 2C). The KEGG pathway enrichment analysis results showed that the differential genes were mainly in the regulation of mRNA metabolic processes, mRNA processing, and RNA splicing pathways (FIG. 2D). These results indicated that the RBP mainly played a role in the metabolism, processing, and splicing of mRNA.

### 6. Acquisition of Prognostic RBP Genes and Construction of Prognostic Risk Models

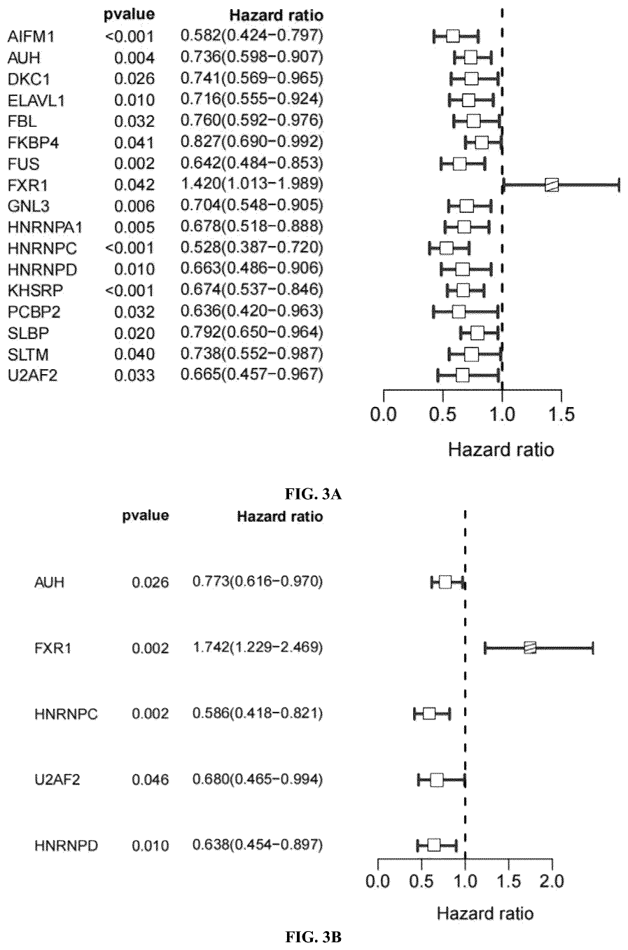

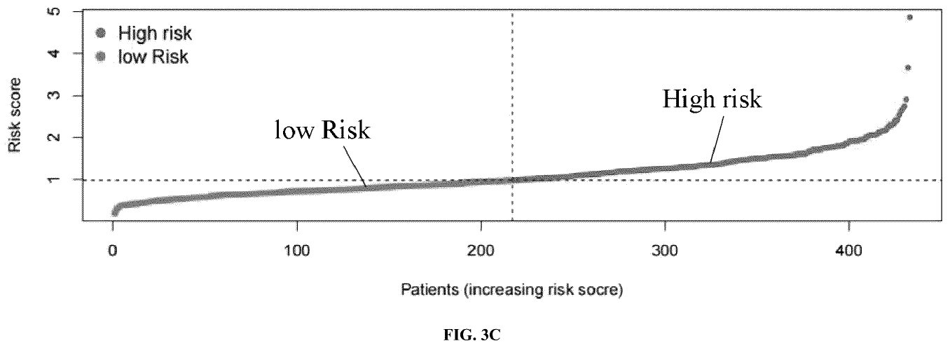

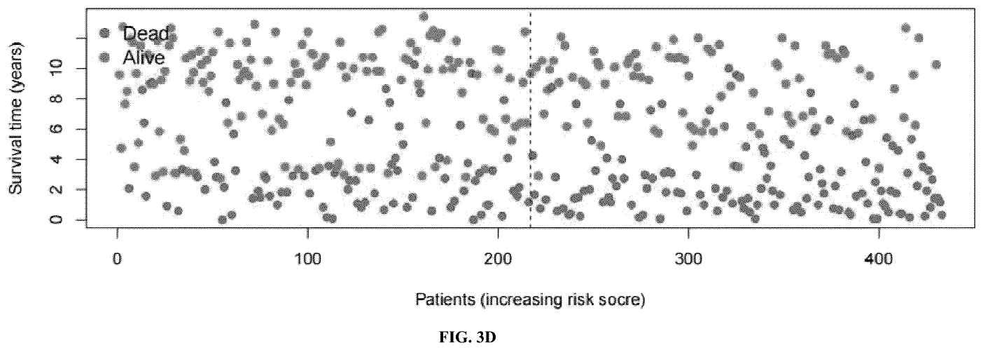

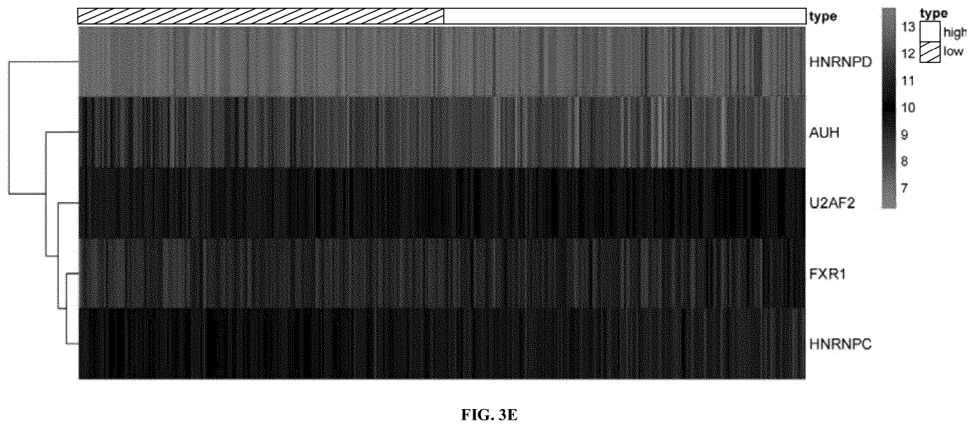

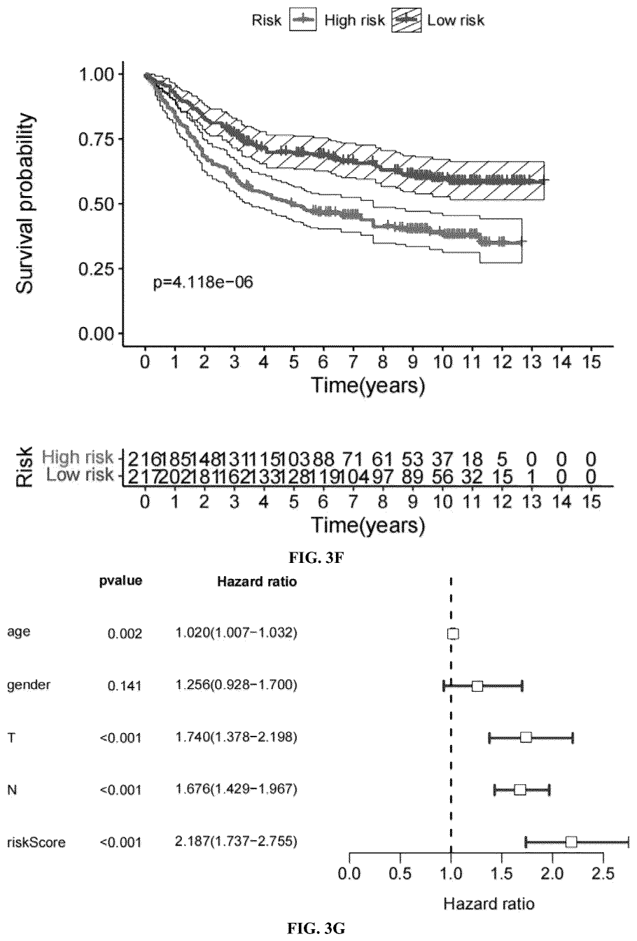

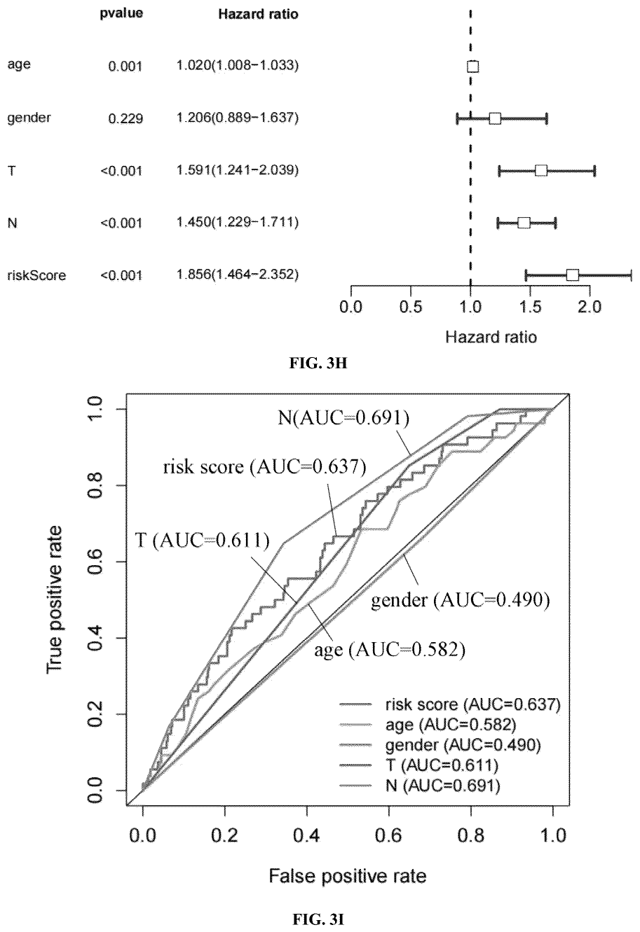

The RBP expression data and clinical information of 433 gastric cancer tissue samples in the gastric cancer-related data set GSE84437 were extracted. Based on the above data and clinical information, the “survival” package of R language was used to conduct univariate COX analysis on the RBP gene binded with the circRNA selected in step 5. Prognosis-related RBP genes were screened with genes of P<0.05, as shown in FIG. 3A. It was found that 17 RBP genes were significantly associated with the prognosis of gastric cancer patients (FIG. 3A). Multivariate COX regression was conducted to analyze the effect of the selected 17 RBP genes on the prognosis of gastric cancer. The genes with P<0.05 were selected as the prognostic RBP genes, as shown in FIG. 3B. 5 RBP genes were identified for risk model construction, where AUH, HNRNPC, HNRNPD, and U2AF2 were low risk factors, and FXR1 was high risk factor. Meanwhile, a regression coefficient (coef) corresponding to each of the 5 RBP genes and related data were obtained in Table 1 based on the multivariate COX analysis.

TABLE 1
Correlation data of 5 RBP genes in multivariate COX analysis

| Gene | Coef | HR | HR.95L | HR.95H | p value |
|---|---|---|---|---|---|
| AUH | −0.257471966977500 | 0.773003295628532 | 0.616279352864140 | 0.969583180542315 | 0.02593679314782750 |
| HNRNPC | −0.534453864372080 | 0.585989232368292 | 0.418458031185980 | 0.820592161843265 | 0.00186531029078691 |
| HNRNPD | −0.449372753941403 | 0.638028226826409 | 0.453972326747102 | 0.896706680656390 | 0.00965858532991128 |
| U2AF2 | −0.386102544595670 | 0.679700822460522 | 0.464997926459776 | 0.993538211171949 | 0.04621391821554370 |
| FXR1 | 0.554848138295511 | 1.741676470732290 | 1.228764808173860 | 2.468687993441690 | 0.00182431439033332 |

Based on the multivariate COX analysis, a risk model was constructed according to the expression level of the prognostic RBP gene and the regression coefficient corresponding to the prognostic RBP gene. The Risk score of each gastric cancer tissue sample was calculated. A calculation method of the Risk score was shown in formula (2): Risk score=(−0.25747×expression level of AUH)+(−0.53445×expression level of HNRNPC)+(−0.44937×expression level of HNRNPD)+(−0.3861×expression level of U2AF2)+(0.554848×expression level ofFXR1).

Based on the Risk score of each sample of the 433 gastric cancer tissues, a median value was calculated to be 0.98541 for each sample of the gastric cancer tissues, and each sample of the gastric cancer tissues was classified into a high-risk group and a low-risk group according to the median value. Based on the risk value data file obtained after multivariate COX analysis, a risk curve was drawn. The analysis results of the risk curve showed that the number of dead patients in the high-risk group was more than that in the low-risk group, and their survival time was relatively shortened (FIG. 3C to FIG. 3D). Based on the risk value data files and the expression data files of 5 key genes obtained after multivariate COX analysis, the expression of 5 key genes in the high-risk group and low-risk group was analyzed. It was found that the expression levels of AUH, HNRNPC, HNRNPD, and U2AF2 were higher in the low-risk group. However, the expression level of FXR1 was higher in the high-risk group. This result was consistent with the multivariate COX analysis (FIG. 3E).

Kaplan-Meier survival analysis was conducted to compare the overall survival (OS) difference between the high-risk group and the low-risk group obtained by the above risk model, and P<0.05 was selected as a cutoff value. The results of Kaplan-Meier survival curve analysis showed that the survival time of patients with low risk score was significantly longer than that of patients with high risk score. Moreover, the 3-, 5-, and 10-year survival rates of patients with low-risk scores were significantly higher than those with high-risk scores (FIG. 3F).

### 7. Independent Prognostic Analysis and Validation of Risk Models

Based on the Risk score obtained in the risk model and the clinical data of the patient, the univariate and multivariate independent prognostic analysis were conducted using the R language “survival” package, and the ROC analysis was conducted using the R language “survivalROC” package; a ROC curve was plotted for the risk prediction model and clinical characteristics including age, gender, T staging, and N staging; and an accuracy of the risk prediction model for a prognosis in predicting the prognosis of a gastric cancer patient was evaluated according to an AUC. In addition, the “survminer” and “survival” packages of the R language were used to analyze the relationship between the age, gender, T staging, and N staging and the survival of patients with gastric cancer, and to verify the predictive ability of the risk model constructed in predicting the survival of patients with different ages, genders, T staging, and N staging.

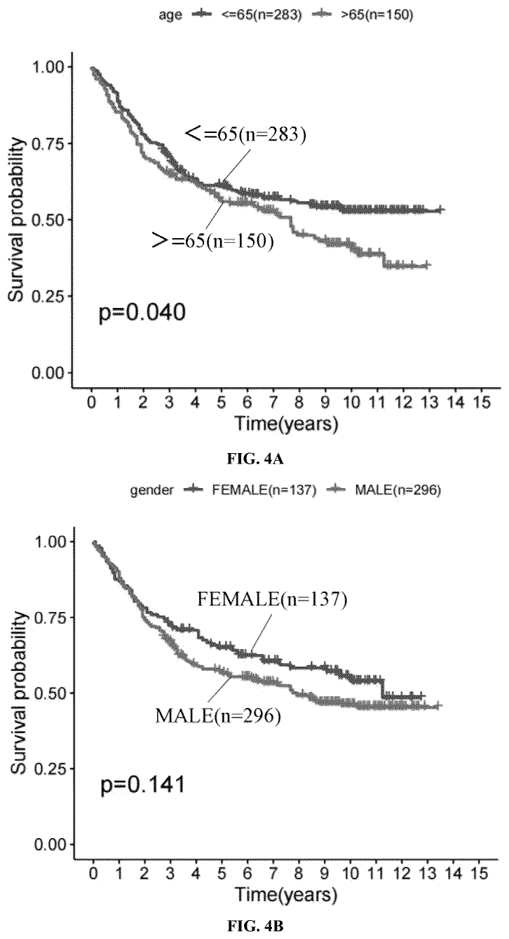

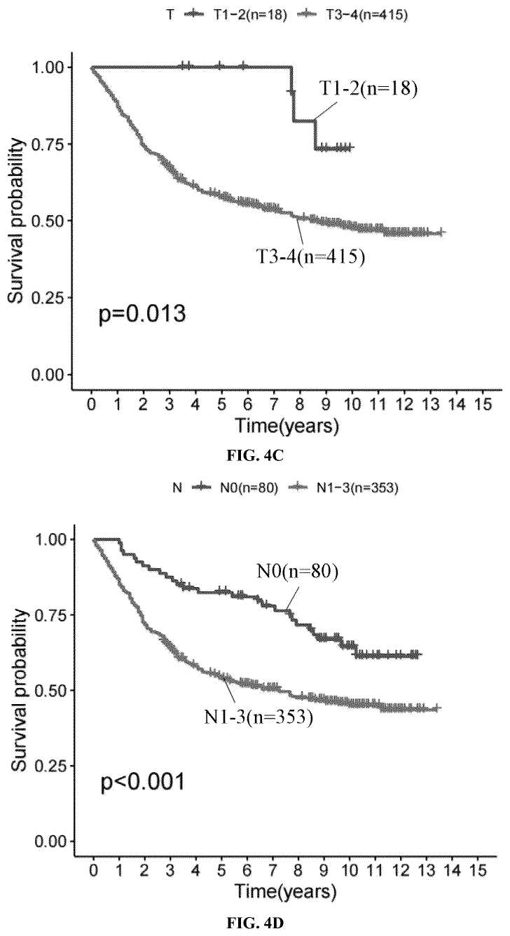

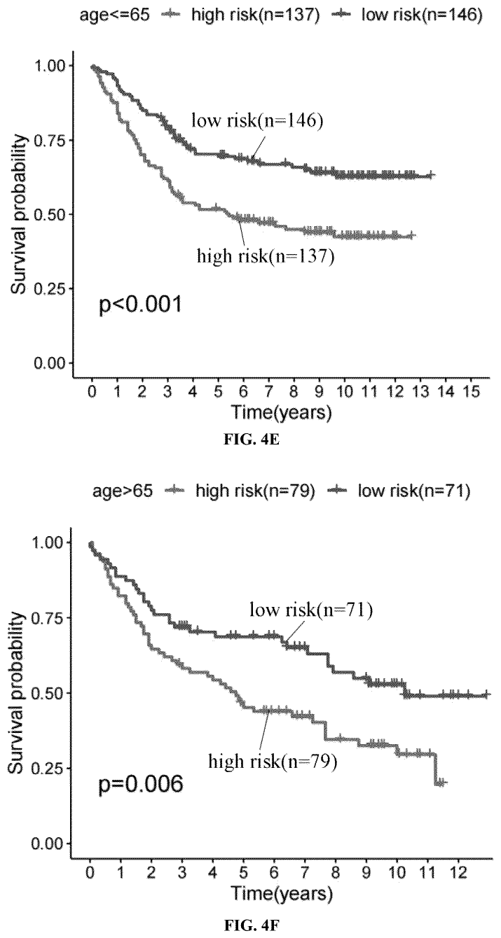

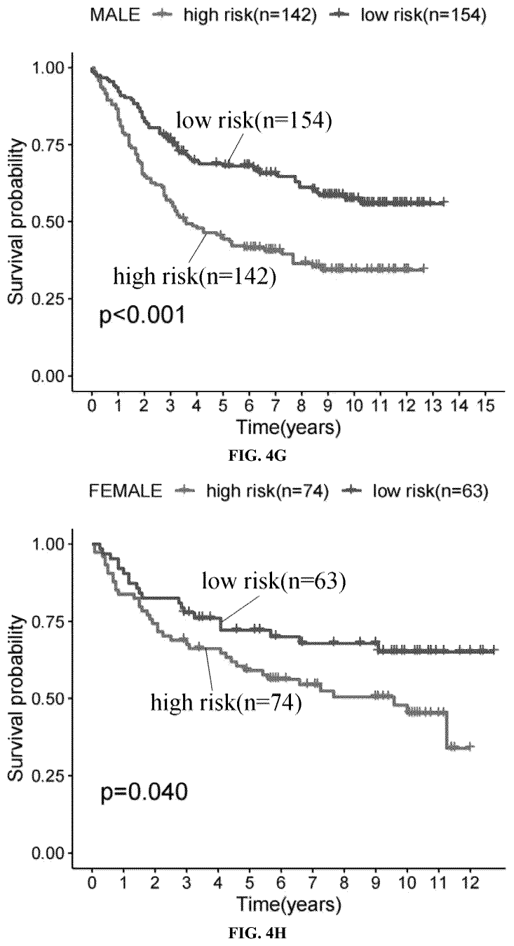

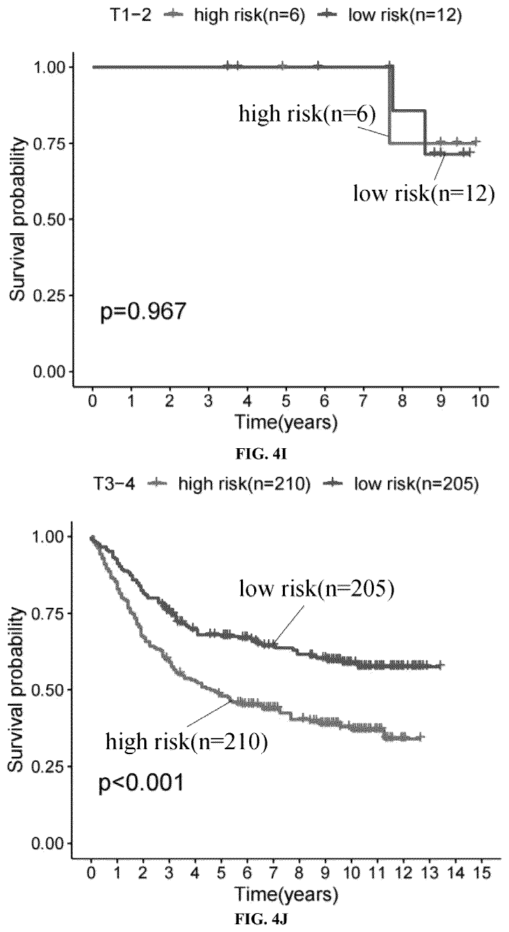

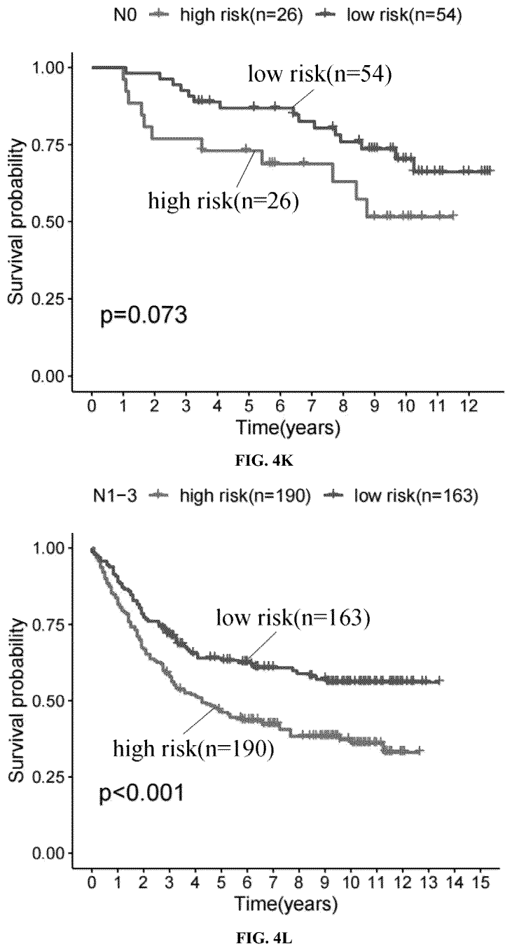

It was found that the constructed risk model could predict the prognosis of gastric cancer patients independently of other factors, and the age, T staging, and N staging could also independently predict the prognosis of gastric cancer patients (FIG. 3G to FIG. 3H). The results of ROC curve analysis showed that the AUC of the constructed risk model was greater than that of age, gender, and T staging, indicating that the model had higher accuracy in predicting the prognosis of gastric cancer patients than that of the age, gender, and T staging (FIG. 3I). In addition, the results of survival analysis of clinical characteristics found that the age, T staging, and N staging were significantly correlated with the survival of patients (FIGS. 4A-D). The clinical traits were further grouped to verify the applicability of the constructed risk model in different populations. The results showed that the risk model could predict the survival of patients with gastric cancer in different age groups (>65 and <=65), different genders, different N staging (NO and N1 to N3), and T3 to T4 stages (FIGS. 4E-L).

### 8. Statistical Analysis

All the above data were processed by graphPad Prism 8.0, and the measurement data were expressed in the form of mean±standard deviation (Mean±SD). The unpaired t-test was used for the comparison between two groups, and the one-way analysis of variance (ANOVA) was used for the comparison between multiple groups. The homogeneity of variances was tested by Levene's method, and Dunnett's t and LSD-t tests were conducted for pairwise comparisons when the variances were homogeneous. Dunnett's T3 test was conducted when variances were not homogeneous. The correlation of gene expression was analyzed by Spearman method. P<0.05 indicated that the difference between the two groups was statistically significant.

In conclusion: the prognostic risk model constructed based on 5 key RBP genes (AUH, HNRNPC, HNRNPD, U2AF2, and FXR1) can better predict the prognosis of patients with gastric cancer.

The above are merely preferred implementations of the present disclosure. It should be noted that several improvements and modifications may further be made by a person of ordinary skill in the art without departing from the principle of the present disclosure, and such improvements and modifications should also be deemed as falling within the protection scope of the present disclosure.

What is claimed is:

1. A construction method of a risk prediction model for a prognosis of gastric cancer, comprising the following steps:
(1) collecting differentially-expressed circular RNAs (circRNAs) from gastric cancer cell-derived exosomes and gastric cancer tissues;
(2) screening an RNA binding protein (RBP) gene that is differentially expressed in the gastric cancer tissues and targetedly binded with the differentially-expressed circRNAs;
(3) obtaining a prognostic RBP gene with a screening criterion of p value<0.05 for the differentially-expressed RBP gene and according to the differentially-expressed RBP gene and a proportional hazards (Cox) regression model;
(4) calculating a Risk score of each sample of the gastric cancer tissues according to an expression level of the prognostic RBP gene and a regression coefficient corresponding to the prognostic RBP gene; and
(5) based on the Risk score of each sample of the gastric cancer tissues, calculating a median value for each sample of the gastric cancer tissues, and classifying each sample of the gastric cancer tissues into a high-risk group and a low-risk group according to the median value.

2. The construction method according to claim 1, wherein the prognostic RBP gene comprises one or more ofAUH, HNRNPC, HNRNPD, U2AF2, and FXR1.

3. The construction method according to claim 1, wherein a process of collecting the differentially-expressed circRNAs comprises: screening the differentially-expressed circRNAs in the gastric cancer cell-derived exosomes from a data set GSE202538 and the gastric cancer tissues from a data set GSE83521 with screening criteria of |log FC|>1 and p value<0.05 for same differential genes.

4. The construction method according to claim 1, wherein a process of screening the differentially-expressed RBP gene that is targetedly binded with the differentially-expressed circRNAs comprises:
(1) predicting RBPs that are targetedly binded with the circRNAs through three databases of Starbase, CSCD, and Circinteractome, and taking a union of the RBPs in the three databases to obtain an RBP that is targetedly binded with the circRNAs; and
(2) screening the RBP that is targetedly binded with the circRNAs obtained in step (1) in a data set of a gastric cancer transcriptome of a database TCGA, and screening the differentially-expressed RBP gene with screening criteria of |log FC|>0.8 and p value<0.05 for a differential gene.

5. The construction method according to claim 1, wherein a calculation method of the Risk score is shown in formula (1) as follows:
$$Risk score = {\sum }_{i = 1}^{n} {coef}_{i} \times {exp}_{i} ,$$
wherein
n represents a number of the prognostic RBP gene; coefᵢ represents a regression coefficient of a prognostic RBP gene i; and expᵢ represents an expression level of the prognostic RBP gene i.

6. The construction method according to claim 1, wherein if the Risk score is less than the median value, a test sample of the gastric cancer tissues is of low risk, indicating that a gastric cancer patient has a desirable prognosis; and if the Risk score is greater than or equal to the median value, the test sample of the gastric cancer tissues is of high risk, indicating that the gastric cancer patient has a poor prognosis.

7. The construction method according to claim 1, wherein based on the Risk score obtained in the risk prediction model and clinical data of a patient, a receiver operator characteristic (ROC) curve is plotted for the risk prediction model and clinical characteristics comprising age, gender, T staging, and N staging; and an accuracy of the risk prediction model for a prognosis in predicting the prognosis of a gastric cancer patient is evaluated according to an area under the curve (AUC).
